(19) United States
(12) Patent Application Publication
(10) Pub. No.: US 2016/0121104 A1
(43) Pub. Date: May 5, 2016

(54) SURGICAL DEVICE

(71) Applicant: Henry Ford Health System, Detroit, MI (US)

(72) Inventor: Stephen Bartol, Windsor (CA)

(21) Appl. No.: 14/931,114

(22) Filed: Nov. 3, 2015

Related U.S. Application Data
(60) Provisional application No. 62/074,150, filed on Nov. 3, 2014.

(51) Int. Cl. A61N 1/05; A61N 1/36; A61B 17/32

(52) U.S. Cl. CPC A61N 1/0551; A61B 17/32002; A61N 1/36

(57) ABSTRACT

A surgical device for use in performing a surgical procedure on a patient. The surgical device comprises an elongate stem having a proximal end, a distal end spaced from the proximal end, a passageway therein extending at least partially between the proximal and distal ends, and an opening extending the length of the passageway to provide visual access into the passageway. The device further comprises a handle disposed at the proximal end portion of the stem, and one or more stimulation channels each terminating at one or more stimulation surfaces of the stem, wherein each of the stimulation surfaces is configured to emit electrical current therefrom to stimulate one or more anatomical structure(s) in proximity thereto.

### CROSS REFERENCE TO RELATED APPLICATIONS

This application claims the benefit of U.S. Provisional Application No. 62/074,150 filed Nov. 3, 2014, the entire contents of which are hereby incorporated by reference.

### TECHNICAL FIELD

The present disclosure relates generally to surgical devices and particularly, but not exclusively, to surgical devices for treating conditions such as spinal stenosis.

### BACKGROUND

Spinal stenosis is an abnormal narrowing of the spinal column and/or openings called foramina through which nerves exit the spinal column. The narrowing of the spinal canal and/or foramina may cause, for example, pressure to be applied to the spinal cord and/or nerves located therein. A patient suffering from spinal stenosis may experience or exhibit one or more of numbness, cramping, weakness, or pain in the legs, back, shoulders, neck, and/or arms, among other symptoms.

One way in which spinal stenosis may be treated is by performing a surgical procedure known as a decompression procedure. In such a procedure impinging bone and/or soft tissue is removed (e.g., cut or shaved) from the spinal canal or foramen in which an affected nerve is located. As a result, the spinal canal or foramen is widened and the pressure on the spinal cord or nerve is relieved.

Conventional devices or tools used to perform decompression procedures are not without their shortcomings, however. For example, during a decompression procedure, devices such as Kerrison rongeurs may not reach far enough into the foramen which may result in inadequate decompression. Additionally, devices such as these cut from the “outside in” removing excessive bone and/or tissue (e.g., the overlying facet joints) which may lead to instability. And instability may, in turn, result in the need for additional—and much more extensive—surgical procedures such as spinal fusion to correct for the instability.

While other conventional devices may overcome at least some of these shortcomings, these devices have their own shortcomings. For example, they may have inadequate nerve detecting capability, require the physician to effectively perform the decompression procedure blind (which may result in, for example, excess bleeding and lack of precision in locating the point at which the decompression is to be performed and/or in the positioning of the device), and may not be configured for use in both lateral (i.e., intervertebral foramen) and central (i.e., spinal canal) decompression procedures.

### SUMMARY

According to one embodiment, there is provided a surgical device for use in performing a surgical procedure on a patient. In an embodiment, the surgical tool comprises an elongate stem having a proximal end, a distal end spaced from the proximal end, and a passageway therein extending at least partially between the proximal and distal ends. The device further comprises one or more stimulation channels each terminating at one or more stimulation surfaces of the stem, wherein each of the stimulation surfaces is configured to emit electrical current therefrom to stimulate one or more anatomical structures in proximity thereto. The surgical tool further comprises a handle disposed at the proximal end portion of the stem. In an embodiment, the surgical tool may be used in the performance of a spinal decompression procedure.

According to another embodiment, there is provided a surgical device for use in performing a surgical procedure on a patient. In an embodiment, the surgical tool comprises an elongate stem having a proximal end, a distal end spaced from the proximal end, and a passageway therein extending at least partially between the proximal and distal ends. The elongate stem further includes an opening extending along the length of the passageway that provides visual access into the passageway, wherein the opening extends more than half the length of the passageway. The surgical tool further comprises a handle disposed at the proximal end portion of the stem. In an embodiment, the surgical tool may be used in the performance of a spinal decompression procedure.

According to yet another embodiment, there is provided a surgical device for use in performing a surgical procedure on a patient. In an embodiment, the surgical tool comprises an elongate stem having a proximal end, a distal end spaced from the proximal end, and a passageway therein extending at least partially between the proximal and distal end portions. The elongate stem further includes an opening extending the length of the passageway that provides visual access into the passageway. The device further comprises a handle disposed at the proximal end of the stem, and one or more stimulation channels each terminating at one or more stimulation surfaces of the stem, wherein each of the stimulation surfaces is configured to emit electrical current therefrom to stimulate one or more anatomical structure(s) in proximity thereto. In an embodiment, the surgical tool may be used in the performance of a spinal decompression procedure.

### BRIEF DESCRIPTION OF DRAWINGS

One or more embodiments of the invention will hereinafter be described in conjunction with the appended drawings, wherein like designations denote like elements, and wherein:

FIG. 1 is a perspective view of an illustrative embodiment of a surgical device;

FIG. 2 is a side elevation view of the surgical device illustrated in FIG. 1 shown with a medical instrument that comprises a part of the surgical device or with which the surgical device is configured to be used;

FIG. 3 is a perspective view of a distal portion of the surgical device shown in FIG. 2;

FIG. 4 is a cross-section view of a distal portion of the surgical device shown in FIG. 2 taken along the line 4-4 in FIG. 32 with the medical instrument shown in FIG. 3 removed;

FIG. 5 is another cross-section view of a distal portion of the surgical device shown in FIG. 2 taken along the line 5-5 in FIG. 3;

FIG. 6 is a perspective view of the cross-section of the distal portion of the surgical device shown in FIG. 5;

FIG. 7 is a perspective view of a distal portion of another embodiment of the surgical device shown in FIG. 2;

FIG. 8 is a perspective view of a distal portion of an illustrative embodiment of the surgical device wherein the surgical device is shown in use;

FIGS. 9 and 10 are diagrammatic views of a distal portion of the surgical device shown in FIG. 1 wherein the surgical device is shown in use; and

FIGS. 11a and 11b are diagrammatic and schematic views of a stimulation source and stimulation channels of an illustrative embodiment of a surgical device, such as, for example, the surgical device(s) shown in one or more of FIGS. 1-10.

### DETAILED DESCRIPTION

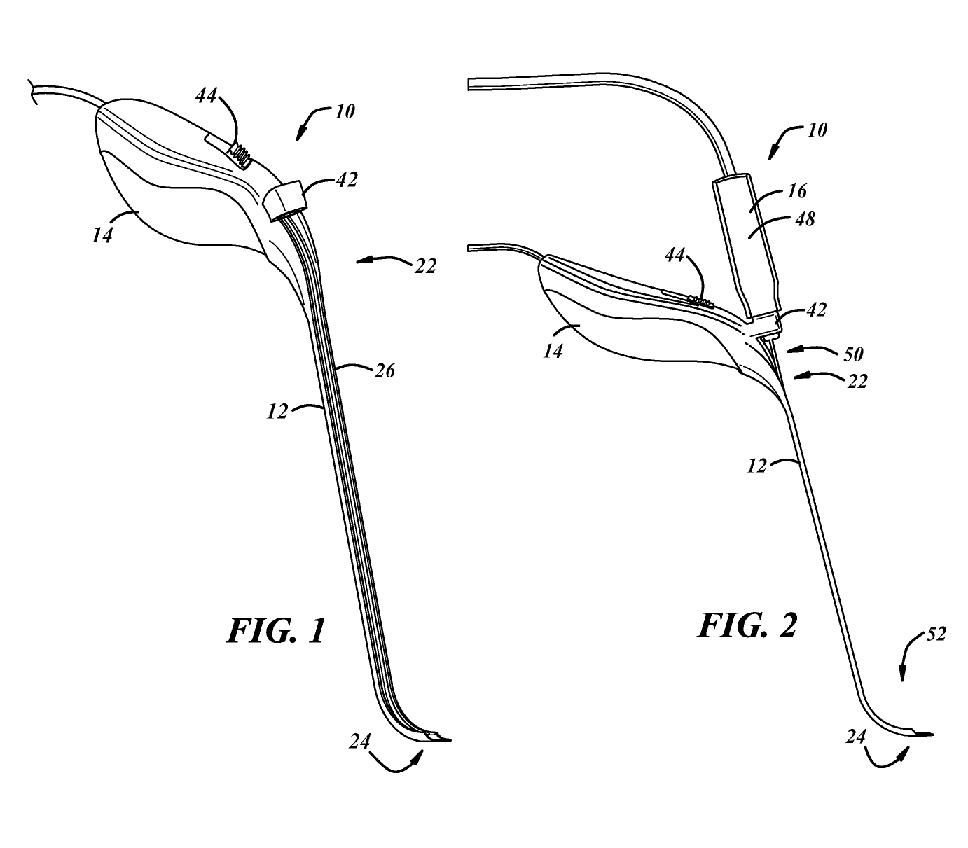

Referring to the drawings, FIG. 1 depicts and illustrative embodiment of a surgical device or tool 10 that may be used for performing one or more surgical procedures on a patient. For purposes of illustration, the description of surgical device 10 below will be with respect to an embodiment wherein the device 10 takes the form of a handheld, minimally-invasive spinal decompression device intended for use in surgical decompression procedures for treating spinal stenosis. It will be appreciated, however, that the present disclosure is not intended to be limited solely to spinal decompression devices or use of the device 10 in the performance of spinal decompression procedures. Rather, in other embodiments, the device 10 may take the form of any number of other types of similar surgical devices or tools intended for use in treating any number of conditions, including, for example and without limitation, cranial nerve decompression and/or peripheral nerve decompression. Additionally, while the description below may be limited to procedures involving humans, it will be appreciated that the embodiments of the surgical device 10 may also be utilized in veterinarian medical procedures involving (non-human) animals as well. Accordingly, the device 10 is not intended to be limited to any particular type(s) of surgical devices, and may find application in the treatment of any number of conditions and/or use in any number of surgical procedures.

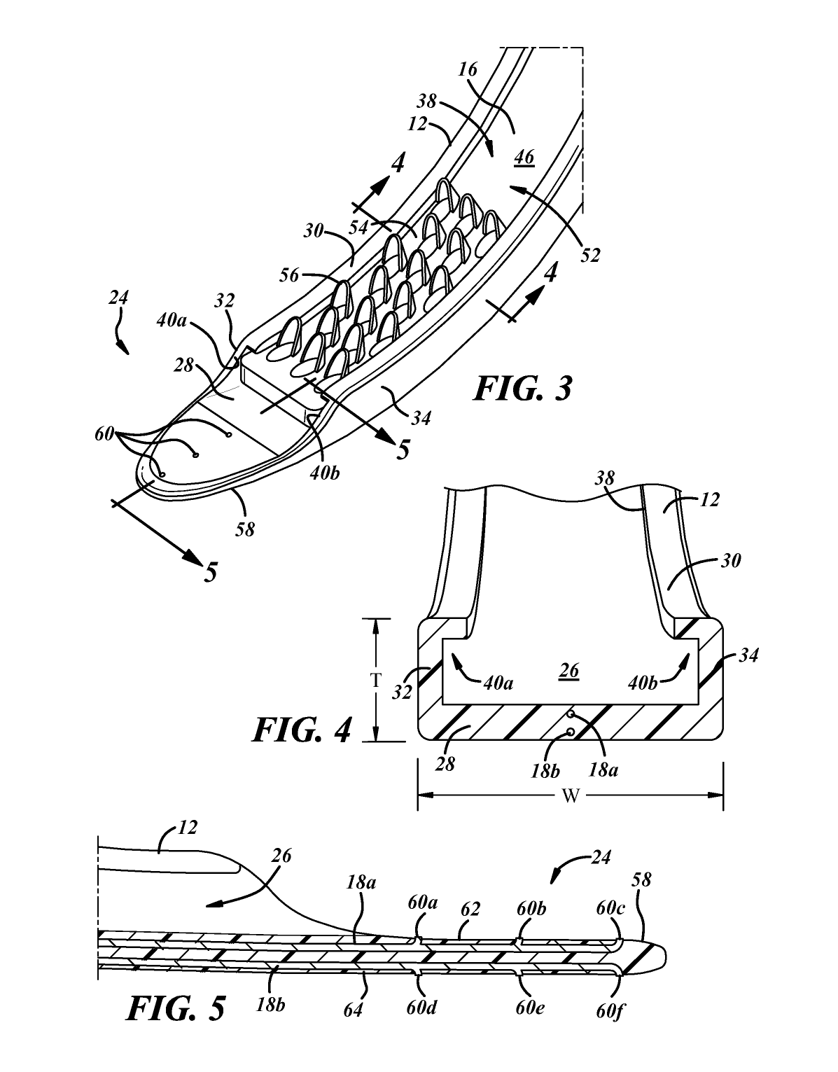

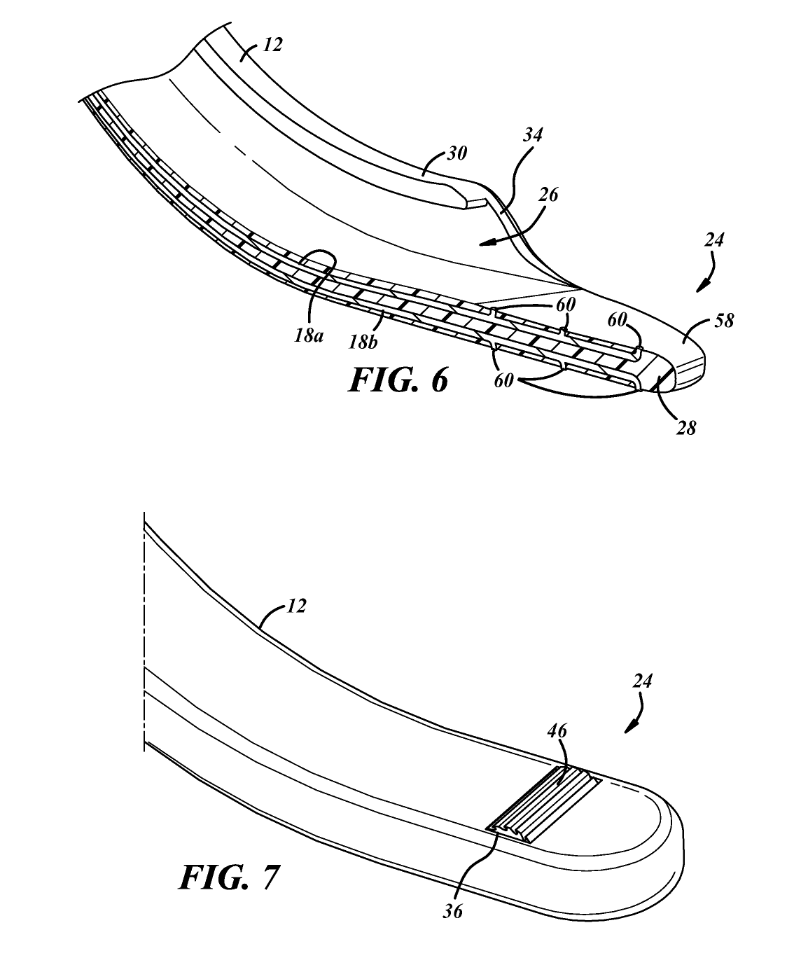

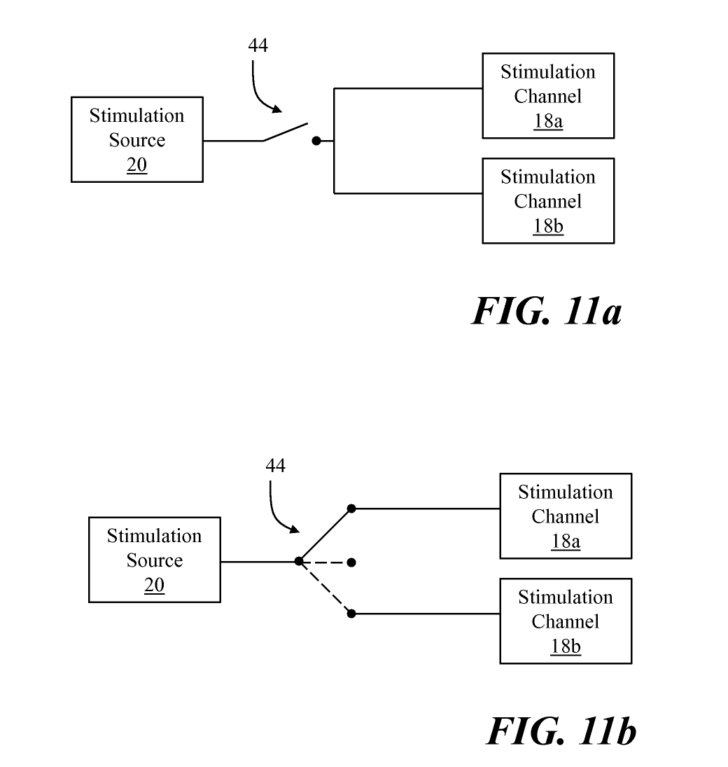

In any event, in the illustrative embodiment, the device 10 includes an elongate stem or shaft (or stem portion) 12 and a handle (or handle portion) 14. As will be described below, and as shown in FIG. 2, the device 10 may further include one or more medical instruments, for example, a cutting or shaving implement or tool 16, one or more stimulation channels 20 (best shown in FIGS. 3-6), and/or a stimulation source (shown diagrammatically in FIGS. 11a and 11b), among potentially other components.

With reference to, for example, FIGS. 1-3, the stem 12 has a proximal end or end portion 22 and a distal end or end portion 24 spaced apart from the proximal end 22, and includes at least one passageway or lumen 26 therein that extends at least partially between the proximal end 22 and the distal end 24. For purposes of this disclosure, “proximal” refers to a direction toward the handle 14 of the device 10, and “distal” refers to a direction away from the handle 14. In some implementations or embodiments, the passageway 26 may be configured (i.e., sized and shaped) to allow one or more medical instruments (e.g., the cutting or shaving implement 16, as will be described below), or at least one or more portions thereon (e.g., a blade of the cutting implement 16) to be inserted or introduced therein and moved or passed to the distal end 24 or to a point near the distal point (e.g., a point closer to the distal-most point of the stem 12 than, for example, the halfway point between the proximal and distal ends 22, 24 of the stem 12) where the tool(s)/device(s) may be used to perform an intended function. In other words, the passageway 26 may be configured to receive at least a portion of one or more medical instruments and to allow that or those portions of the instrument(s) to be moved therein. Accordingly, in such implementations or embodiments, at least a portion of the stem 12 forms or serves as a track for medical instrument(s). In at least some implementations or embodiments, the passageway 26 may be additionally or alternatively configured to house or have disposed therein one or more components that enable at least certain functionality of the device 10. These components may include, for example and without limitation, one or more electrical wires or cables and components required to facilitate the movement of a medical instrument or tool (e.g., cutting implement 16) disposed therein (e.g., drive shafts, gears, linkages, etc.), to cite a few possibilities.

The passageway 26 may be defined or bounded by one or more interior surfaces of the stem 12, and the inner or interior surface(s) of one or more walls that form the stem 12, in particular. For example, in the illustrative embodiment depicted in FIGS. 3 and 4, the stem 12 includes a bottom wall 28, a top wall 30, and a pair of side walls 32, 34 extending between the bottom and top walls 28, 30, with the interior surfaces of the walls 28, 30, 32, 34 combining to the define the passageway 26. In the illustrated embodiment, the walls are arranged such that the stem 12 has a rectangular cross-section; though other cross-sectional shapes (e.g., circular, oval, square, etc.) may certainly be used instead depending on the particular construction or arrangement of the stem 12, and/or the intended use/purpose of the device 10. In any event, the passageway 26 may extend from the proximal end 22 of the stem 12 (and, in an embodiment, the proximal-most point of the stem 12 (i.e., the point directly adjacent the handle 14)) to the distal end 24. In some implementations or embodiments, the passageway 26 may extend to the distal-most point of the stem 12 (i.e., the distal tip); while in other embodiments, it may terminate at a point that is proximal to the distal-most point of the stem 12 (as shown in FIG. 3, for example), but closer to the distal-most point of the stem 12 than, for example, the halfway point between the proximal and distal ends 22, 24 of the stem 12.

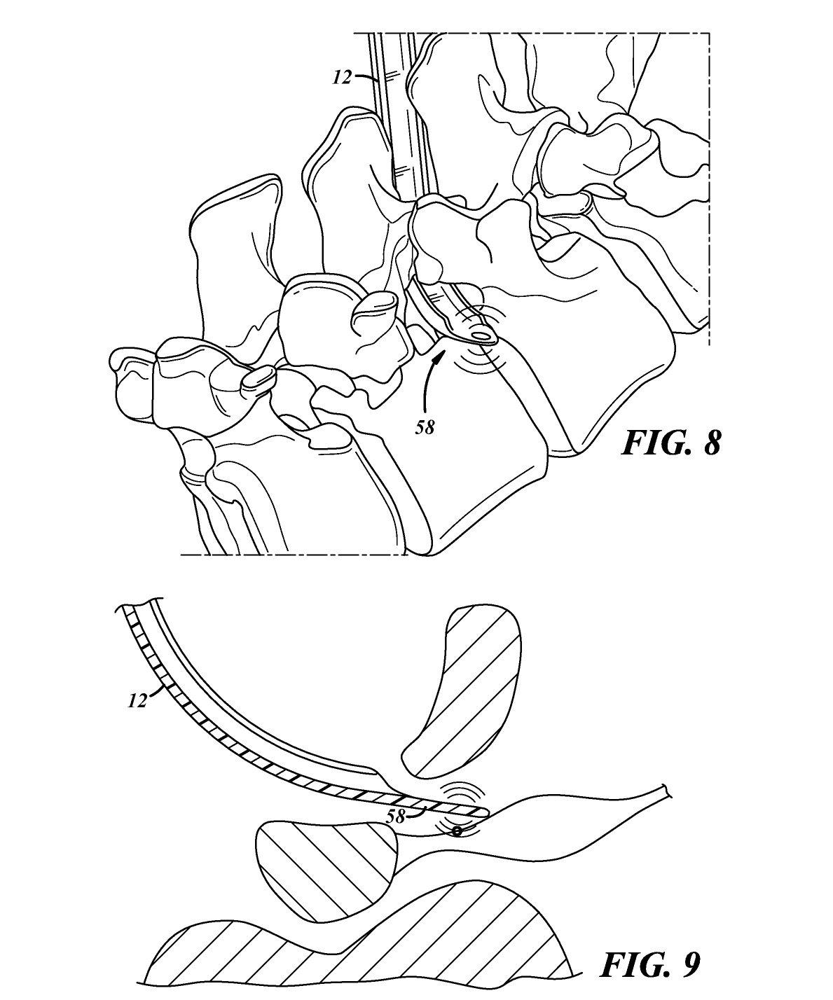

In addition to the passageway 26, the stem 12 may further include one or more openings therein that may serve any number of purposes. For example, stem 12 may include an opening at the proximal end 22 that provides access to the passageway 26 and is configured (e.g., sized and shaped) to allow medical instrument(s) or portion(s) thereof to be inserted into the passageway 26. In certain implementations, such as, for example, that illustrated in FIG. 7 and described in greater detail below, the stem 12 may additionally or alternatively include one or more openings 36 configured to have at least a portion of a medical instrument (e.g., a portion of the cutting implement 16, for example, a rotary blade) mounted or disposed therein. As shown in FIGS. 3 and 4, the stem 12 may also or alternatively include one or more openings providing a window of sorts into the passageway 26 to allow the visualization of a medical instrument as it moves within the passageway 26 to, for example, allow for the precise positioning of the instrument within the passageway 26 (e.g., to allow for a determination to be made that the instrument has reached a desired position at or near the distal end 24 of the stem 12).

By way of illustration, FIGS. 1, 3, and 4 depict the stem 12 having an opening 38 in the top wall 30 thereof that extends the length of the passageway 26 and provides visual access to the interior of the passageway 26. In the illustrated embodiment, the opening 38 allows for the insertion of a medical instrument into the passageway 26 at the proximal end 22, and also allows for the visualization of the instrument as it moves through the entirety of the passageway 26 to a point at or near the distal end 24 (e.g., the endpoint of the passageway 26). In the illustrated embodiment, the opening 38 does not extend all the way to either one of the side walls 32, 34 of the stem 12, which provides the advantage that a pair of grooves 40a, 40b are formed by the partial or discontinuous top wall 30, the bottom wall 28, and the side walls 32, 34, respectively, that serve to retain or capture the medical instrument within the passageway 26 (i.e., the instrument cannot pass through the opening 38 and out of passageway 26, rather it is disposed within the grooves 40a, 40b). In other embodiments, a single groove 40 may be formed that serves the same purpose.

While in the illustrated embodiment only one opening 38 is provided that extends the length of the passageway 26 (e.g., from the proximal end 22 all the way to a point at or near the distal end 24), it will be appreciated that other arrangements are certainly possible. For example, stem 12 may include a plurality of openings 38 that are distributed or spaced longitudinally and/or laterally along the length of the stem. Alternatively, stem 12 may include a single opening that does not extend the entire distance between the proximal and distal ends, but rather is disposed distal to the proximal end 22 and/or proximal to the distal end 24 (i.e., an opening that is disposed along the length of the stem at a location between the proximal and distal ends of the stem). Similarly, in at least some embodiments, the opening 38 may not extend the entire length of the passageway 26, but rather may extend along only a portion thereof (e.g., a majority or more than half of the length but not the entire length). Accordingly, the present disclosure is not limited to any particular number or arrangement of openings in the stem 12.

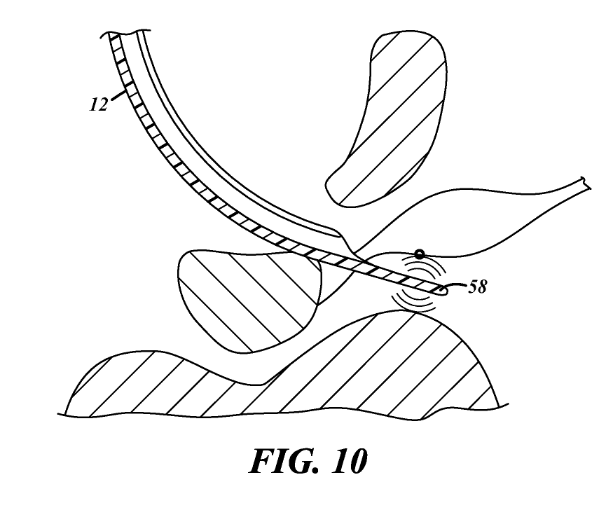

In an embodiment, the stem 12 is rigid, meaning that the stem is inflexible under a predetermined load or force that is typically applied when the device 10 is used for its intended purpose (e.g., in the performance of spinal decompression procedures). In other embodiments, however, the stem 12, or at least certain portion(s) thereof, may be flexible. The stem 12 may be formed of any number of suitable materials, for example, glass fiber, reinforced nylon, polycarbonate, anodized aluminum, and/or carbon fiber, to cite a few possibilities. And for reasons that will be appreciated in view of the description below, the material of which all or portion(s) of the stem 12 is/are formed may be electrically non-conductive. Further, the stem 12 may be sufficiently thin (e.g., from the outer surface of the top wall 30 to the outer surface of the bottom wall 28) and narrow (e.g., from the outer surface of the side wall 32 to the outer surface of the side wall 34) so as to be maneuverable within tight areas, for example, the spinal canal and other foramina (see, for example, FIGS. 8-10). For instance, and with reference to FIG. 4, in one non-limiting example, the stem 12 have a thickness “T” and a width “W” that are suitable to allow for the maneuvering of the stem 12 within the spinal canal and other foramina. In some implementations, the stem 12 may be substantially straight or planar along its length from the proximal-most point thereof to the distal-most point; in other implementations or embodiments, however, the stem 12 may be curved or angled, or may include one or more curved or angled portions. For example, as shown in FIGS. 1 and 2, all or a part of stem adjacent to, and in certain embodiments, including, the distal end 24 may be curved. In the illustrated embodiment, the curved portion extends from a point that is distal to the halfway point of the stem 12 between the proximal- and distal-most endpoints thereof, and proximal to the distal-most endpoint. It will be appreciated, however, that other arrangements of the curved portion are certainly possible.

With reference to FIG. 1, the handle 14 is disposed at the proximal end 22 of the stem 12 and is configured to allow a physician to manipulate the position and orientation of the stem 12, and the distal end 24 thereof, in particular, during a surgical procedure, and to also stabilize the stem 12 while the procedure is being performed. The handle 14 may of a unitary, one-piece construction or may be comprised of multiple pieces that are mechanically coupled together using, for example and without limitation, conventional fasteners (e.g., screws), connectors/fittings (e.g., those for use in interference, press, or snap fit connections/couplings, etc.), adhesives, and/or using other suitable techniques. Similarly, the handle 14 (or at least a portion thereof) may be integrally formed with the stem 12 such that they form a single, unitary piece, or the handle 14 and stem 12 may be separate components that are mechanically coupled together using conventional techniques such as, for example, one or more of those identified above or using other suitable techniques. In an embodiment, the handle 14 may be constructed of the same material(s) as the stem 12 (e.g., glass fiber, reinforced nylon, polycarbonate, anodized aluminum, or carbon fiber, to cite a few possibilities); while in other embodiments different materials may be used. As will be described below and as shown in FIGS. 1 and 2, in some implementations, the handle 14 (or, in another embodiment, the stem 12) may include a mount or bracket 42 for supporting a portion or component of the cutting implement 16 or another medical instrument. More particularly, the mount 42 may be located in close proximity to the stem 12 such that a portion of the relevant instrument may be inserted into the passageway 26 of the stem 12, and may be suitably sized and shaped for receiving and retaining or holding a portion of the relevant device(s) therein.

In some implementations, the handle 14 may include one or more electromechanical ports disposed therein. These ports are configured to allow for the electrical and mechanical connection between components of the device 10 (e.g., the stimulation channels 18 described below) and other components that are part of the device 10 or with which the device 10 is configured to be used (e.g., the stimulation source 20 described below). More particularly, an electromechanical connector of an electrical cable of (or connected to) a component of the device 10 or that may be used with device 10 (e.g., the stimulation source 20) may be plugged into or mated with an electromechanical port disposed in the handle 14. Alternatively, rather than electromechanical port(s) being disposed in the handle 14 itself, one or more cables or wires may extend from the handle 14 and terminate in an electromechanical connector.

As shown in FIGS. 1 and 2, and as will be described in greater detail below, the handle 14 may also include one or more selectors or electrical switches 44 disposed therein or thereon that may be used for a variety of purposes. The switch(es) 44 may comprise one or a combination of: pushbuttons; toggle switches; single pull, single throw switches; single pull, double pull switches; single throw changeover switches; double pull, single throw switches; and double throw, double throw switches, to cite only a few possibilities.

Depending on the implementation, the handle 14 may also have an inner cavity (not shown) in which various components required for the operation of the device 10 may be disposed. These components may include, for example and without limitation, one or more electrical circuits (e.g., matching circuits to match components carried by the stem 12 or handle 14 with other components of the device, electrical wires or cables configured to connect components carried by the stem 12 or handle 14 with electromechanical port(s) and/or switch(es) disposed in or on the handle 14, etc.).

In addition to the components described above, the device 10 may further include one or more medical instruments that may be used in conjunction with the combination of the handle 14 and stem 12. One example of such an instrument is the cutting or shaving tool or implement 16. The implement 16 may take a number of different forms known in the art; however, as shown FIG. 2, it may generally include a blade 46 and a drive assembly 48 that is configured to drive the movement of the blade 46.

In the illustrated embodiment, the implement 16 comprises a reciprocating saw. In this embodiment, the blade 46 has a proximal end 50 and a distal end 52, and may be formed of any number materials, for example, spring steel or other suitable materials. Similar to the stem 12 described above, “proximal” in relation to the cutting implement 16 refers to a direction toward the drive assembly 48 of the implement 16, and “distal” refers to a direction away from the drive assembly 48. In an embodiment, the blade 46 is sufficiently flexible to be inserted into the passageway 26 of the stem 12 and easily passed or moved therein to a point at or near the distal end 24 of the stem 12, including through the curved portion of the stem 12. The blade 46 may include a cutting or working area 54 at or near the distal end 52 that includes, as shown in FIG. 3, one or more teeth 56 suitable for cutting or shaving bone and/or soft tissue.

The drive assembly 48 may comprise any number of types of drive assemblies known in the art, for example, hydraulic, pneumatic, electric (e.g., electric motor), or any other suitable type of drive assembly, and may include an output shaft that is operatively coupled to the blade 46, and the proximal end 50 thereof, in particular, to drive the reciprocation of the blade 46.

In the illustrated embodiment, the implement 16 is a separate component from the handle 14 and stem 12 but is configured to be assembled therewith. More particularly, the blade 46 may be inserted into the passage 26 at or near the proximal end 22 of the stem 12 and passed down to a desired point at or near the distal end 24 thereof, and at least a portion of the drive assembly 48 may be supported by the mount 42. Accordingly, in use, the stem 12 may be inserted into a patient's body and positioned at a location of interest. The blade 46 of the implement 16 may then be inserted into the passageway 26 and advanced therein to move the blade 46, and the working area 54 thereof, in particular, into a desired position, and the drive assembly 48 may be assembled with the mount 42 to support the implement 16. The drive assembly 48 may then be activated to reciprocate the blade 46 within the passageway 26 resulting in the cutting or shaving of anatomical structure(s) of interest.

While the description of implement 16 has thus far been with respect to a reciprocating-type device/tool, it will be appreciated that the present disclosure is not intended to be so limited. Rather, in other embodiments, the implement 16 may comprise other types of cutting or shaving devices/tools known in the art, for example, oscillating or vibrating-types of devices/tools, or any other suitable device/tool. For example, in the embodiment or implementation illustrated in FIG. 7, the implement 16 may comprise a rotatable cutting or shaving device/tool. More specifically, rather than the blade 46 being a reciprocating blade as described above, it may comprise a rotatable blade. In this embodiment, the blade 46 may be mounted in or carried by the stem 12, for example, in an opening in the stem 12, for example, the opening 36; though other arrangements are certainly possible. As with the embodiment described above, the drive assembly 48 may comprise any number of types of drive assemblies and, as will be appreciated by those having ordinary skill in the art, may include an output as well as one or more components or linkages between the output and the blade 46 to translate motion generated by the drive assembly 48 to the rotatable blade 46. The particular types of components or linkages required will depend on the particular type of drive means being used, but may include, for example and without limitation, a flex shaft, one or more gears, and the like. In an embodiment, these components or linkages may be housed within the passageway 26. Additionally, while only one blade is shown in FIG. 7 and is depicted as having a particular orientation or axis of rotation that is transverse to the centerline or longitudinal axis of the stem 12, in other embodiments, multiple blades and/or different orientations or arrangements may be used.

In the embodiments described above, the implement 16 is separate and distinct from both the handle 14 and stem 12. It will be appreciated, however, that in other embodiments or implementations, the implement 16 may be integrated into the handle 14 and stem 12. More particularly, the drive assembly 48, or at least portions thereof, may be integrated into the handle 14. Accordingly, it will be appreciated that the present disclosure is not intended to be limited to any particular construction or arrangement of the constituent components of the device 10, and particularly, the implement 16, handle 14, and stem 12.

While the description relating to the implement 16 has thus far been with respect to the implement comprising a constituent part of the device 10, in other embodiments, the device 10 may not include the implement 16 (or any other medical instrument, for that matter), but rather may be simply configured for use with such an instrument. In other words, a commercial embodiment of the device 10 may not include the implement 16 or any other medical instrument with which the device 10 may be used to perform one or more medical (surgical) procedures.

In addition to those components described thus far above, and as illustrated in FIGS. 4-6, device 10 may further include one or more integrated stimulation channels 18, and in the illustrated embodiment, a first stimulation channel 18a and a second stimulation channel 18b. As will be described in greater detail below, each stimulation channel 18 can be used to stimulate anatomical structures, for example, bone, tissue, nerves, etc., in sufficiently close proximity to the device 10. This stimulation may be used, for example, for nerve detection and/or monitoring. Each stimulation channel 18 may be comprised of one or more electrical conductors, for example, one or more wires. The conductor(s) may be disposed in or on the stem 12 and, in an embodiment such as that shown in FIGS. 4-6, may be embedded in a wall of the stem 12 (e.g., the bottom wall 28, in the illustrated embodiment). In an instance wherein there are multiple channels 18, the conductor(s) of each channel are insulated from the conductor(s) of the other channel(s) by, for example, the material(s) forming the stem 12 and/or insulation layers surrounding the conductor(s) of the respective channels.

In some implementations or embodiments, the conductor(s) of the channels 18 may extend from an electromechanical port or connector that is part of or associated with the handle 14 (e.g., those described above with respect to handle 14) and that is configured to be electrically connected to the output of a stimulation source (e.g., current generator). In an embodiment, the conductor(s) may extend from that electromechanical port, through at least a portion of the handle 14 and the stem 12, and may then terminate at one or more stimulation areas 58 located in or on the stem 12, and at one or more stimulation surfaces 60 in the stimulation area(s) 58, in particular. For purposes of illustration, the description below will be with respect to an embodiment such as that illustrated in FIGS. 3, 5, and 6 wherein the device 10 includes a single stimulation area 58 that is disposed or located at the distal-most point of the stem 12 (i.e., at the distal tip of the device that is distal of the passageway 26), and that is an integral and contiguous part of the bottom wall 28 of the stem 12. It will be appreciated, however, that in other embodiments, other arrangements are certainly possible. For example, instead of forming part of the bottom wall 28 of the stem 12, the stimulation area 58 may be part of the top wall 30 and/or one or both of side walls 32, 34, or may be separate from the top, bottom, or side walls altogether. Further, instead of being disposed at the distal-most point of the stem 12, the stimulation area 58 may be located anywhere along the length of the stem 12. Still further, instead of including a single stimulation area 58, the device 10 may include a plurality of stimulation areas wherein adjacent areas may be longitudinally and/or laterally spaced apart from each other. Accordingly, it will be appreciated that the present disclosure is not intended to be limited to any particular number or arrangement of stimulation area(s) 58.

The stimulation area 58 includes one or more stimulation surfaces 60 each of which corresponds to one of the stimulation channel(s) 18. For example, in the embodiment illustrated in FIGS. 3, 5, and 6, the stimulation area 58 includes six (6) stimulation surfaces 60 (i.e., 60a-60f)—three (3) (i.e., surfaces 60a-60c) corresponding to the first stimulation channel 18a, and three (3) (i.e., surfaces 60d-60f) corresponding to the second stimulation channel 18b. More particularly, in the illustrated embodiment, the stimulation area 58 includes a top or first side 62 and a bottom or second side 64, with the top side 62 including the stimulation surfaces 60a-60c corresponding to stimulation channel 18a, and the bottom side 64 including the stimulation surfaces 60d-60f corresponding to stimulation channel 18b. It will be appreciated, however, that in other embodiments the stimulation area 58 may have a different number and/or arrangement of stimulation surfaces, and therefore, the present disclosure is not intended to be limited to any particular number or arrangement.

The stimulation surface(s) 60 may take a number of forms, including, for example and without limitation, the terminal end of a conductor of the corresponding stimulation channel 18 (as shown in FIGS. 3, 5, and 6) or a conductive pad or electrode electrically connected to one or more conductor(s) of the corresponding stimulation channel 18. As illustrated in FIGS. 8-10, the stimulation surface(s) 60 is/are configured and operative to emit electrical current when the stimulation channel 18 associated therewith is electrically coupled to the stimulation source 20 of or that is being used in conjunction with the device 10, and the source 20 is active or “on” such that electrical current is applied to that particular channel 18 (i.e., when the channel 18 is energized). The emitted current is intended to stimulate anatomical structures (e.g., bone, tissue, nerves, etc.) in close proximity (e.g., adjacent) to the stimulation area 58 and stimulation surface(s) 60 from which the current is emitted for purposes of performing nerve detection and/or monitoring (e.g., mono-polar nerving detection/monitoring) functionality. For purposes of this disclosure, “proximity” is intended to mean roughly the area in which the surgical procedure is to be performed (e.g., the area of surgical decompression), and may be, for example, within 0-15 mm of the stimulation area 58, and in an illustrative embodiment, within 1-6 mm of the stimulation area 58.

It will be appreciated that in an embodiment such as that illustrated in FIGS. 3, 5, and 6 wherein the stimulation area 58 has stimulation surfaces 60 facing in different directions, current may be emitted in those different directions in order to stimulate anatomical structures in different locations adjacent to those surfaces 60 (e.g., above, below, left, right, etc.). In an embodiment, the device 10 is not itself configured to perform the actual nerve detection/evaluation functionality; rather device 10 is only configured to provide the stimulation required for it to be performed. In such an embodiment, the actual nerve detecting/monitoring functionality would be performed by a device other than device 10, and as such, a description of the nerve detection/monitoring functionality will not be provided. It will be appreciated that any number of nerve detection/monitoring modalities or technologies may be used, for example, mechanomyography (MMG) and electromyography (EMG), and as such, the present disclosure is not limited to the use of any particular type(s).

In an embodiment, the stimulation channel(s) 18 of the device 10 may be automatically energized whenever the channel(s) 18 is/are electrically connected to the stimulation source 20 and the source is active or “on.” Alternatively, the channel(s) 18 may be selectively energized via a switch or selector (e.g., one or more of switches 44) disposed, for example, in or on the handle 14, and electrically connected between the channel(s) 18 and the source 20. In other words, one or more switches 44 may be configured to selectively control the supply of current from the stimulation source 20 to the channel(s) 18. More particularly, in an embodiment such as that illustrated in FIG. 11a, when the switch 44 is in a first position, the channel(s) 18 may be disconnected from the source 20, and when the switch 44 is in a second position, the channel(s) 18 may be connected to the source 20. In an embodiment wherein the device 10 includes multiple stimulation channels 18, a switch or selector (e.g., one or more of switches 44) may be used to selectively energize at least one, but less than all, of the channels 18. For example, in an embodiment such as that illustrated in FIG. 11b wherein the device 10 includes first and second channels 18a, 18b, switch 44 may have a first position in which the first channel 18a is electrically connected to the source 20 and the second channel 18b is disconnected therefrom, and a second position in which the second channel 18b is electrically connected to the source 20 and the first channel 18a is disconnected. As shown in FIG. 11b, in an embodiment, the switch 44 may have a third position (i.e., a neutral position) in which neither of the channels 18a, 18b are electrically connected to the source 20, rather both are disconnected.

In another embodiment, rather than the device 10 having one or more stimulation channels 18 integrated within the stem 12 as described above, the device 10 may include a separate stimulation probe (not shown) that may be electrically connected to the stimulation source 20 and that may used to perform the stimulation functionality described above. In such an embodiment, the probe may include one or more stimulation channels and stimulation areas that operate in the same manner as the channel(s) 18 and area(s) 58 described above, the only difference being that when stimulation is needed, the probe is inserted into and passed down the passageway 26 in the stem 12 in the same or similar manner as cutting implement 16 described above. Once appropriately positioned, the stimulation functionality may be performed, and upon completion, the stimulation probe may be removed and, if appropriate, replaced with the implement 16 or another medical instrument. Accordingly, it will be appreciated that the device 10 may be configured to perform the stimulation functionality in a number of ways, and thus, the present disclosure is not intended to be limited to any particular way(s) of doing so.

Another component that the device 10 may include, or with which the device 10 may be configured to be used, is a protective sheath (not shown). The sheath may be inserted into the patient and navigated to a desired location or position, and then the stem 12 of the device 10 may be inserted into a passageway or lumen in the sheath and advanced to the desired location. Alternatively, the sheath and the stem 12 may be inserted into the patient together at the same time with all or a portion of the stem 12 being disposed within a passageway or lumen of the sheath. When the device is positioned and oriented in a desired way, the distal end 24 of the stem 12 may then be advanced out of the sheath so that the required functionality may be performed. In either instance, and as will be appreciated by those having ordinary skill in the art, the purpose of the sheath is to protect anatomical structures, for example, organs, tissue, nerves, etc. as the device 10 is being navigated within a patient's body and during a medical procedure performed using the device 10. For example, the sheath may protect anatomical structures from being inadvertently cut or contacted by the blade 46 of the cutting implement 16, or another portion or component of the device 10.

For purposes of illustration, an example of a procedure that may be performed using the device 10 described above will now be provided. It will be appreciated, however, that this is just one example and that the device 10 may find application in any number of other procedures in addition to that described below. First, the physician exposes the spinal canal by performing a hemilaminotomy and removing ligamentum flavum. The stem 12 of the device 10 is then inserted or introduced into the spinal canal and positioned in the foramen or under the lamina to be decompressed. Nerve detection is then performed using the stimulation channel 18b and the stimulation surfaces 60 corresponding thereto located on the underside 64 of the stimulation area 58 and MMG (or EMG) technologies; a positive response being indicative of the proximity of a nerve. The nerve detection process is then repeated using the stimulation channel 18a and the stimulation surfaces 60 corresponding thereto located on the top side 62 of the stimulation area 58. If the level of current required to obtain a positive response at the top side (e.g., the cutting surface) is greater than that required to obtain a positive response on underside, then it can be determined that it is safe to proceed with cutting. In instance wherein a reciprocating type cutting implement is used, the blade 46 of the implement 16 is then introduced or inserted into the passageway 26 of the stem 12. When the working or cutting area 54 of the blade 46 is appropriately positioned, bone and/or soft tissue may be cut to enlarge the opening of the foramen. The stem 12 may be advanced into the foramen as needed to further enlarge the opening. Once the physician determines that the foramen has been sufficiently or adequately enlarged, nerve detecting testing can once again be performed to test the integrity of the nerve. Adequacy of the decompression that was performed may be assessed or determined by (1) the ease with which the stem 12 is inserted and removed from the foramen, (2) palpation with a second feeling tool (such as a Woodson elevator), (3) direct visualization, and/or (4) with fluoroscopic imaging. The physician may have the further option of testing the health of the nerve to determine if the amount of decompression is adequate. This may be done by repeating the nerve detection testing through the stimulation channel(s) 18 and comparing the results with pre-decompression testing.

It will be appreciated in view of the foregoing that at least some of the embodiments of the present disclosure have the benefits or advantages, among possibly others, that the device is configured to be inserted into, for example, the central or vertebral canal or foramen, as well as lateral and/or neural foramen, and may then be used to perform a decompression device from the “inside out” by removing impinging and soft tissue from inside the canal or foramen (as opposed to having to perform the procedure “outside in”, as is done using some conventional devices). Additionally, in at least some implementations or embodiments, the device may include integrated stimulation channels that may used to selectively stimulate anatomical structures to enable nerve detection/monitoring functionality to ensure, for example, that nerves are located a safe distance away from the cutting implement 16 during a decompression procedure.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A surgical device for use in performing a surgical procedure on a patient, comprising:
an elongate stem having
a proximal end,
a distal end spaced from the proximal end, and
a passageway therein extending at least partially between the proximal and distal ends;
one or more stimulation channels each terminating at one or more stimulation surfaces of the stem, wherein each of the stimulation surface is configured to emit electrical current therefrom to stimulate one or more anatomical structure(s) in proximity thereto; and
a handle disposed at the proximal end of the stem.

2. The surgical device of claim 1, wherein the passageway is configured to receive at least a portion of a medical instrument, and to allow the at least a portion of the medical instrument to be moved therein.

3. The surgical device of claim 2, wherein the medical instrument comprises a cutting implement, and the at least portion of the medical instrument comprises a blade of the cutting implement.

4. The surgical device of claim 1, wherein the stem includes at least one groove in the passageway thereof configured to retain at least a portion of a medical instrument when the at least a portion of the medical instrument is disposed within the passageway.

5. The surgical device of claim 1, wherein a first portion of the stem is straight and a second portion of the stem is curved.

6. The surgical device of claim 1, wherein the handle comprises a bracket configured to support a medical instrument used in conjunction with the device.

7. The surgical device of claim 1, further comprising a stimulation source configured to be electrically connected to each of the one or more stimulation channels.

8. The surgical device of claim 1, further comprising one or more electrical switches each electrically connected to at least one of the stimulation channels, and each configured to selectively control the supply of electrical current from a stimulation source to at least one of the one or more stimulation channels.

9. The surgical device of claim 1, further comprising a cutting implement having a blade and a drive assembly configured to drive movement of the blade, wherein the passageway of the stem is configured to receive at least a portion of the blade and to allow the at least a portion of the blade to move therein.

10. The surgical device of claim 9, wherein the blade comprises an elongate flexible blade having a proximal end and a distal end spaced from the proximal end and including a cutting area disposed at the distal end thereof, wherein the passageway of the stem is configured to allow the blade to be reciprocated therein by the drive assembly.

11. The surgical device of claim 9, wherein the blade comprises a rotatable blade.

12. The surgical device of claim 1, wherein the surgical procedure is a spinal decompression procedure.

13. The surgical device of claim 1, wherein the stem further comprises one or more openings extending along the length of the passageway that provides visual access into the passageway of the stem.

14. A surgical device for use in performing a surgical procedure on a patient, comprising:
an elongate stem having
a proximal end,
a distal end spaced from the proximal end,
a passageway therein extending at least partially between the proximal and distal ends, and
an opening extending along the length of the passageway that provides visual access into the passageway, wherein the opening extends more than half of the length of the passageway; and
a handle disposed at the proximal end of the stem.

15. The surgical device of claim 14, wherein the opening of the stem extends the length of the passageway.

16. The surgical device of claim 14, further comprising one or more stimulation channels each terminating at one or more stimulation surfaces of the stem, wherein each of the stimulation surface is configured to emit electrical current therefrom to stimulate one or more anatomical structure(s) in proximity thereto.

17. The surgical device of claim 16, further comprising a stimulation source configured to be electrically connected to each of the one or more stimulation channels.

18. The surgical device of claim 16, further comprising one or more electrical switches each electrically connected to at least one of the one or more stimulation channels, and each configured to selectively control the supply of electrical current from a stimulation source to at least one of the one or more stimulation channels.

19. The surgical device of claim 14, wherein the passageway is configured to receive at least a portion of a medical instrument and to allow the at least a portion of the medical instrument to be moved therein.

20. The surgical device of claim 14, wherein the stem includes at least one groove in the passageway thereof configured to retain at least a portion of a medical instrument when the at least a portion of the medical instrument is disposed within the passageway.

21. The surgical device of claim 14, wherein the handle comprises a bracket configured to support a medical instrument used in conjunction with the device.

22. The surgical device of claim 14, further comprising a cutting implement having a blade and a drive assembly configured to drive movement of the blade, wherein the passageway of the stem is configured to receive at least a portion of the blade and to allow the at least a portion of the blade to move therein.

23. The surgical device of claim 14, wherein the surgical procedure is a spinal decompression procedure.

24. A surgical device for use in performing a surgical procedure on a patient, comprising:
an elongate stem having
a proximal end,
a distal end spaced from the proximal end,
a passageway therein extending at least partially between the proximal and distal ends, and
an opening extending the length of the passageway that provides visual access into the passageway;
a handle disposed at the proximal end of the stem; and
one or more stimulation channels each terminating at one or more stimulation surfaces of the stem, wherein each of the stimulation surfaces is configured to emit electrical current therefrom to stimulate one or more anatomical structure(s) in proximity thereto.
